(19) United States
(12) Patent Application Publication
(10) Pub. No.: US 2015/0257787 A1
(43) Pub. Date: Sep. 17, 2015

(54) SUBCUTANEOUS SENSOR IMPLANT INSERTION TOOL WITH BIORESORBABLE TIP

(71) Applicant: Senseonics, Incorporated, Germantown, MD (US)

(72) Inventors: Casey Haigh, Germantown, MD (US); Jeremy Emken, Germantown, MD (US); Philip Huffstetler, Germantown, MD (US)

(73) Assignee: Senseonics, Incorporated, Germantown, MD (US)

(21) Appl. No.: 14/657,205

(22) Filed: Mar. 13, 2015

Related U.S. Application Data
(60) Provisional application No. 61/952,962, filed on Mar. 14, 2014.

(51) Int. Cl. A61B 17/34; A61B 5/145; A61L 31/16; A61L 31/14; A61L 31/04

(52) U.S. Cl. CPC A61B 17/3468; A61L 31/148; A61L 31/048; A61L 31/16; A61B 5/14503; A61L 2300/412; A61L 2300/216; A61L 2300/426; A61B 2017/00004

(57) ABSTRACT

A system, method, and apparatus for implanting a sensor or other item into tissue of an animal includes the use of a tapered tip, which may be made from a bioresorbable material. The tapered tip may be removably attached to the distal end of a sleeve or cannula within which the item to be implanted is positioned. The tapered tip may facilitate advancement of the sleeve into the tissue to form a pocket within the tissue. Retraction of the sleeve may remove the item from the sleeve and separate the tip from the sleeve, thereby depositing the tip and the item in the pocket formed in the tissue.

### CROSS-REFERENCE TO RELATED APPLICATION

The present application claims the benefit of priority to U.S. Provisional Application Ser. No. 61/952,962, filed on Mar. 14, 2014, which is incorporated herein by reference in its entirety.

### FIELD OF THE INVENTION

This invention relates to sensor implantation devices and, more specifically, to a tip for placement at the end of tool for implantation of an implantable sensor whereby the tip facilitates insertion of the tool and a sensor carried therein and wherein, upon deployment of the sensor and after removal of the tool, the tip and the sensor remain implanted.

### BACKGROUND

An implantable sensor for continuous monitoring of an analyte of interest (e.g., glucose, oxygen, cardiac markers, low-density lipoprotein (LDL), high-density lipoprotein (HDL), triglycerides, etc.) may be implanted in a living animal (e.g., a living human). The sensor may be implanted, for example, in a living animal's arm, wrist, leg, abdomen, peritoneum, or other region of the living animal suitable for sensor implantation. The sensor may be implanted beneath the skin (e.g., in the subcutaneous tissues or peritoneal space).

Current insertion tools for implanting an implantable sensor involve the use of two independent tools, a tunneling tool, such as a blunt dissector, and an insertion tool, that are used to place a subcutaneous implanted sensor. A small slit is made through the epidermis, and, to create a subcutaneous pocket, the blunt dissector is advanced into the slit. After the dissector is inserted up to a specified depth, it is removed and the pocket formed by the dissector collapses. The insertion tool, which includes a retractable sleeve or cannula with a sensor loaded into a chamber at a distal end of the sleeve or cannula, is then advanced into the pocket to a specified depth. The insertion tool is then actuated, for example, through a pull back of a thumb slider which is connected to a sleeve or cannula covering and/or protecting the sensor. This translation reveals and deploys the underlying sensor into the previously created pocket as the insertion tool is withdrawn from the pocket.

Thus, the current protocol involves the use of two separate and independent tools and the reopening of a semi-collapsed subcutaneous pocket with the second advancement of the sensor-loaded insertion tool. Moreover, while the blunted cone of the blunt dissector is able to easily expand and advance into tissue, the flat distal end of the cannula on the insertion tool, dulled slightly by a small protrusion of the sensor, may catch on the wound opening (i.e., the small slit) and may cause additional trauma on the reopening of the subcutaneous pocket.

Accordingly, there is a need for improvements in procedures and instruments for implanting items into the body.

### SUMMARY

Aspects of the invention are embodied in a bioresorbable and/or biodegradable conical tip that is shaped like the tip of a blunt dissector and can be placed over the distal end of the sensor and adjacent and into the distal end of the cannula of an insertion tool. The tip may enable a current insertion tool to be used as both a blunt dissector for opening a subcutaneous pocket and an insertion tool for deploying the sensor into the pocket. Upon removal of the insertion tool, the tip and the sensor remain in the pocket. In some embodiments, the tip is made from a bioresorbable and/or biodegradable material, and the tip will eventually dissolve within the pocket.

Thus, in accordance with aspects of the present invention, the procedure for implanting an implantable sensor may utilize a single tool to both form the subcutaneous pocket and deploy the sensor, and both the tip and the sensor may be deployed and remain in the body of the subject (i.e., in the pocket).

By effectively combining the blunt dissector tool and the insertion tool into a single tool for implanting the sensor, some embodiments of the invention may reduce the number of tools required for a sensor implantation procedure and may reduce the procedural complexity of the surgical implantation by reducing the number of insertions, the surgical time, and tissue trauma.

One aspect of the invention may provide an apparatus for implanting an item in animal tissue. The apparatus may include a tubular sleeve, a tapered tip, and an actuating mechanism. The tubular sleeve may include a compartment at a distal end of the sleeve. The compartment may be configured to receive the item to be implanted such that the item is disposed within the sleeve. The tapered tip may be configured to be removably attached to the distal end of the sleeve and to facilitate advancement of an assembly including the sleeve and the tip into or through the animal tissue. The actuating mechanism may be coupled to the sleeve and may be configured to retract the sleeve with respect to the item disposed within the sleeve to thereby remove the item from the sleeve and separate the tip from the distal end of the sleeve.

In some embodiments, the sleeve may be made from stainless steel or polytetrafluoroethylene (PTFE). In some embodiments, the tip may have a conical shape. In some embodiments, the tip may have a rounded tip. In some embodiments, the tip may have a ring projecting from an end thereof, and the ring may be configured to be inserted into the distal end of the sleeve to retain the tip to the sleeve by a friction fit. In some embodiments, the tip may be configured to be removably attached to the distal end of the sleeve by clipping or adhering the tip to the sleeve.

In some embodiments, the item may include a sensor configured to detect an analyte or substance of interest in the animal tissue. In some embodiments, the tip may be at least partially hollow. In some embodiments, the tip may be formed from a material that is bioresorbable or biodegradable. In some embodiments, the tip may be made from a polymer comprising one or more materials selected from the group consisting of lactic acid, glycolic acid, glucose, polytrimethylene carbonate, collagen, laminin, hydroxyapatite, hyaluronan, and amino acids. In some embodiments, the material of which the tip is made may further include an immune suppressant. In some embodiments, the immune suppressant may include poly-salicylate or a corticosteroid. In some embodiments, the material of which the tip is made may further comprise angiogenic factors to promote vascular growth and healing.

In some embodiments, the tip may be made from a polymer comprising one or more materials selected from the group consisting of polycaprolactone (PCL), poly(ester-ether)s, poly(amino acid)s, and poly(anhydride)s. In some embodiments, the poly(ester-ether)s may include polydioxanone (PDX). In some embodiments, the poly(amino acid)s may include poly(L-glutamate), poly(L-lysine, or poly(L-leucine). In some embodiments, the poly(anhydride)s may include poly(sebacic acid) or poly(sebacic acid-co-carboxyphenoxypropane).

Another aspect of the invention may provide a tip. The tip may be used in connection with a device for implanting an item in animal tissue. The device may include a tubular sleeve and an actuating mechanism. The tubular sleeve may include a compartment at a distal end of the sleeve. The compartment may be configured to receive the item to be implanted such that the item is disposed within the sleeve. The actuating mechanism may be coupled to the sleeve and configured to retract the sleeve with respect to the item disposed within the sleeve to thereby remove the item from the sleeve. The tip may include a tapered end and a connecting end opposite the tapered end. The connecting end may be configured to enable the tip to be removably attached to the distal end of the sleeve. The tip may be configured such that, when the actuating mechanism retracts the sleeve with respect to the item, the retraction removes the item from the sleeve and also separates the tip from the distal end of the sleeve. The tip may be formed from a material that is bioresorbable or biodegradable.

In some embodiments, the connecting end may include a ring projecting from the end of the tip opposite the tapered end, and the ring may be configured to be inserted into the distal end of the sleeve to retain the tip to the sleeve by a friction fit. In some embodiments, the tip may be configured to be removably attached to the distal end of the sleeve by clipping or adhering the tip to the sleeve. In some embodiments, the tip may have a conical shape. In some embodiments, the tip may have a rounded tip. In some embodiments, the tip may be at least partially hollow.

In some embodiments, the tip may be made from a polymer including one or more materials selected from the group consisting of lactic acid, glycolic acid, glucose, polytrimethylene carbonate, collagen, laminin, hydroxyapatite, hyaluronan, and amino acids. In some embodiments, the tip may be made from a polymer including one or more materials selected from the group consisting of polycaprolactone (PCL), poly(ester-ether)s, poly(amino acid)s, and poly(anhydride)s. In some embodiments, the poly(ester-ether)s may include polydioxanone (PDX). In some embodiments, the poly(amino acid)s may include poly(L-glutamate), poly(L-lysine, or poly(L-leucine). In some embodiments, the poly(anhydride)s may include poly(sebacic acid) or poly(sebacic acid-co-carboxyphenoxypropane).

In some embodiments, the material of which the tip is made may further include an immune suppressant. In some embodiments, the immune suppressant may include poly-salicylate or a corticosteroid. In some embodiments, the material of which the tip is made may further include angiogenic factors to promote vascular growth and healing.

Still another aspect of the invention may provide a method for implanting an item in animal tissue. The method may include placing the item within a tubular sleeve at a distal end of the sleeve. The method may include removably attaching a tapered tip to the distal end of the sleeve. The method may include inserting the tip into an incision formed in the animal tissue. The method may include advancing an assembly comprising the tip and the sleeve with the item disposed therein into the animal tissue, thereby forming a pocket within the animal tissue. The method may include effecting a retraction of the sleeve with respect to the item within the pocket, thereby removing the item from the sleeve and separating the tip from the distal end of the sleeve and depositing the item and the tip in the pocket within the animal tissue.

In some embodiments, the tapered tip may be made of a material that is bioresorbable or biodegradable.

Other features and characteristics of the present invention, as well as the methods of operation, functions of related elements of structure and the combination of parts will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The accompanying drawings, which are incorporated herein and form part of the specification, illustrate various, non-limiting embodiments of the present invention. In the drawings, common reference numbers indicate identical or functionally similar elements.

FIGS. 1A and 1B illustrate a side view of a sensor holder and a perspective view of a sensor, respectively.

FIGS. 2A-2C illustrate perspective, top, and side views of a tunneling tool (e.g., a blunt dissector).

FIGS. 3A-3C illustrate perspective, top, and side views of an insertion tool.

FIG. 4 illustrates the creation of a subcutaneous pocket for a sensor using a tunneling tool.

FIG. 5 illustrates the subcutaneous implantation of a sensor by an insertion tool.

FIG. 6 illustrates a perspective view of a bioresorbable tip embodying aspects of the invention.

FIG. 7 illustrates a perspective view of the bioresorbable tip mounted onto the cannula of a sensor insertion tool.

FIG. 8 illustrates a transverse cross-sectional view of the bioresorbable tip mounted onto the sleeve, or cannula, of a sensor insertion tool.

### DETAILED DESCRIPTION OF THE INVENTION

This description may use relative spatial and/or orientation terms in describing the position and/or orientation of a component, apparatus, location, feature, or a portion thereof. Unless specifically stated, or otherwise dictated by the context of the description, such terms, including, without limitation, top, bottom, above, below, under, on top of, upper, lower, left of, right of, in front of, behind, next to, adjacent, between, horizontal, vertical, diagonal, longitudinal, transverse, etc., are used for convenience in referring to such component, apparatus, location, feature, or a portion thereof in the drawings and are not intended to be limiting.

Furthermore, unless otherwise stated, any specific dimensions mentioned in this description are merely representative of an exemplary implementation of a device embodying aspects of the invention and are not intended to be limiting. Unless otherwise indicated or the context suggests otherwise, as used herein, “a” or “an” means “at least one” or “one or more.”

In general, the present invention may be implemented in conjunction with any implantable sensor or other component that can be implanted by the two-step procedure generally described in the BACKGROUND section above and in more detail below.

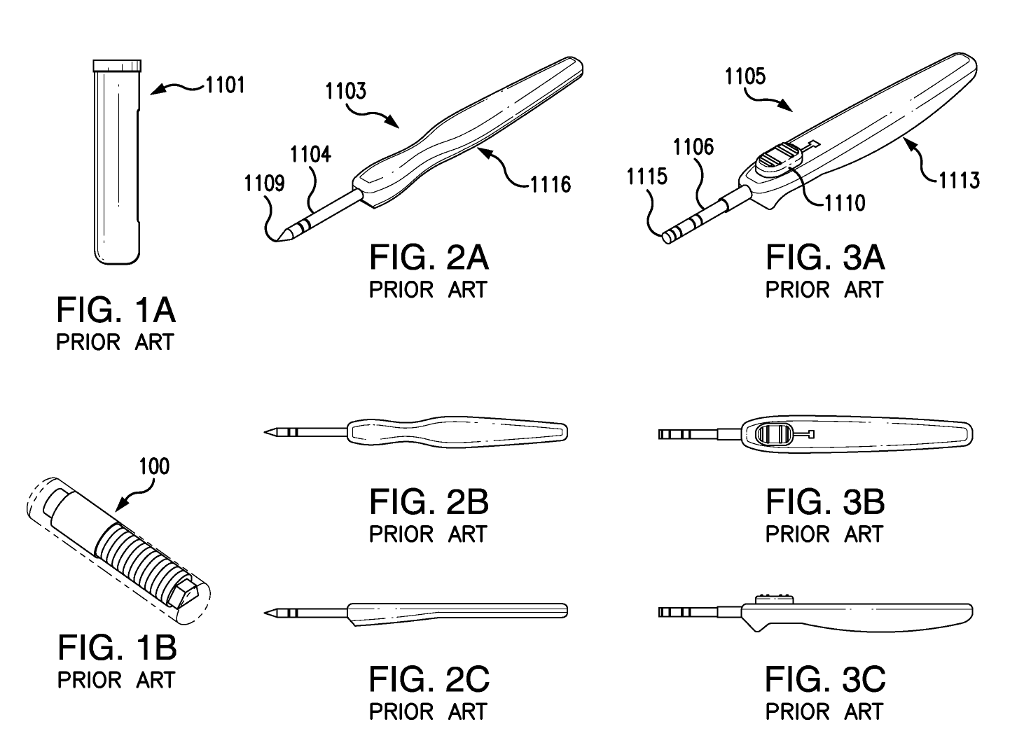

By way of example, FIG. 1B shows an implantable sensor 100, which may be an optical sensor (e.g., a fluorometer) and may be a chemical or biochemical sensor. The sensor 100 is configured to be implanted in a living animal (e.g., a living human) and may, for example, be configured for continuously monitoring an analyte of interest (e.g., glucose, oxygen, cardiac markers, low-density lipoprotein (LDL), high-density lipoprotein (HDL), triglycerides, etc.) in a subcutaneous region of the living animal.

The sensor 100 may be implanted, for example, in the living animal's arm, wrist, leg, abdomen, peritoneum, or other region of the living animal suitable for sensor implantation. The sensor 100 may be implanted beneath the skin (i.e., in the subcutaneous or peritoneal tissues). In some embodiments, the sensor 100 may be implanted subcutaneously (e.g., in a location of the body that is appropriate for subcutaneous measurement of interstitial fluid glucose), and no portion of the sensor 100 protrudes from the skin.

In accordance with current procedures and protocols, sensor 100 may be subcutaneously implanted using a tunneling tool 1103, such as a blunt dissector, as shown in FIGS. 2A-2C and an insertion tool 1105 as shown in FIGS. 3A-3C. As illustrated in FIG. 1A, the procedure may also include the use of a sensor holder 1101, which may hold the sensor 100 (e.g., in sterile and/or lubricated condition) before implantation.

Sensor 100 may have a small and relatively cylindrical shape, and, as shown in FIGS. 2A-2C, the tunneling tool 1103 may include a rod 1104 having a blunted, conical tip 1109 projecting from a handle 1116.

As illustrated in FIGS. 3A-3C, the insertion tool 1105 may be an implantation device that is shaped similarly to the tunneling tool 1103 and includes a handle 1113 and a tubular sleeve, or cannula, 1106 (e.g., stainless steel or polytetrafluoroethylene (PTFE) tube) that extends from the handle 1113 and includes a compartment at a distal end 1115 configured to receive and hold the sensor 100 for implantation into the patient. The sleeve 1106 and the handle 1113 are configured so that the sleeve 1106 can be retracted into handle 1113. A mechanism may be provided to enable a user to effect a retraction of the sleeve 1106. For example, a slide button 1110 on the handle 1113 may be coupled to the sleeve 1106 so that the sleeve 1106 can be retracted or extended by sliding the button 1110 back or forward relative to the end of the handle 1113 from which the sleeve 1106 protrudes.

After the insertion tool 1105 is inserted into a desired location of the patient the sliding button 1110 operatively coupled to the sleeve 1106 may be pulled back to retract the sleeve 1106 to release the sensor 100 into a desired location on the patient, and the implantation tool 1105 is removed.

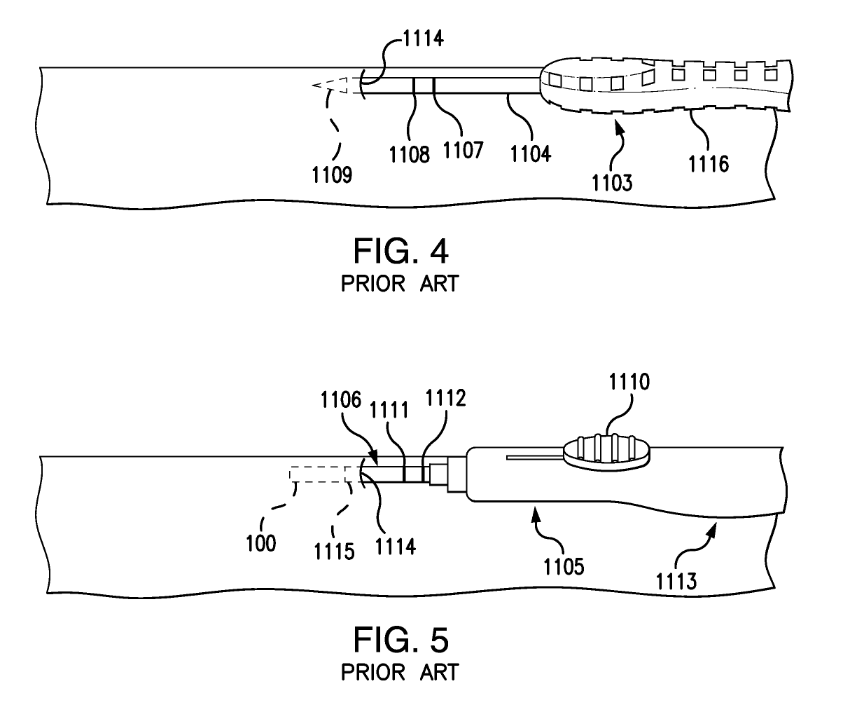

As shown in FIG. 4, initially, a small incision 1114 is made in the epidermis with a blade scalpel. The tip 1109 of the tunneling tool 1103 is inserted into the slit, and the blunt tip 1109 of rod 1104 is advanced into the tissue to create a pocket for the sensor 100. In some non-limiting embodiments, the pocket may be immediately subcutaneous (e.g., no more than 1-2 cm below the surface of the skin), and the pocket may follow the fascial plane at the bottom of the subcutaneous layer. One or more indicators, such as bands 1107, 1108, may be provided on the rod 1104 of the tunneling tool 1103 for gaging the depth of insertion of the rod 1104 into the tissue.

The insertion tool 1105 is prepared for inserting the sensor into the pocket by moving the button 1110 forward and placing a sensor 100 into the compartment at the distal end 1115 of the sleeve 1106.

As shown in FIG. 5, after the rod 1104 of the tunneling tool 1103 is withdrawn from the just-formed pocket, the insertion tool 1105 is used to deliver the sensor 100 into the pocket. The sleeve 1106 of the insertion tool 1105 is inserted into the pocked by a specified amount. One or more indicators, such as bands 1111, 1112, may be provided on the sleeve 1106 of the insertion tool 1105 for gaging the depth of insertion of the sleeve 1106 into the pocket. Once the sleeve 1106 of the insertion tool 1105 is inserted to the specified depth, the button 110 is moved back, which retracts the sleeve 1106 from around the sensor 100, thereby depositing the sensor 100 in the pocket created by the tunneling tool 1103.

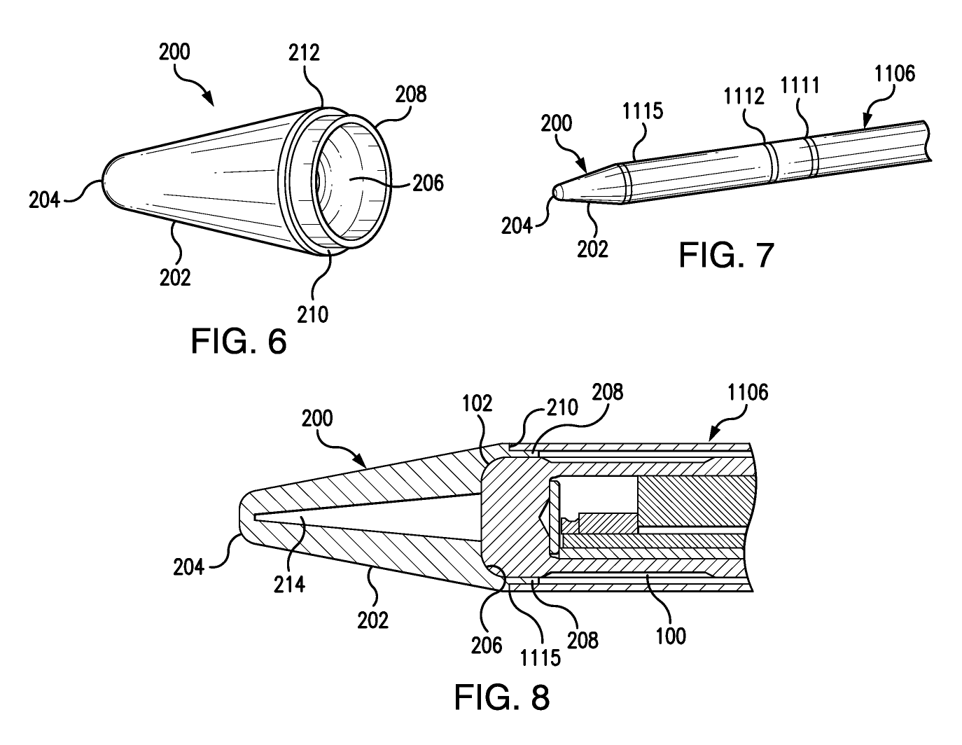

A tip embodying aspects of the present invention is represented by reference number 200 in FIG. 6. In some embodiments, the tip 200 is configured to be placed on the distal end of the sleeve (e.g., sleeve 1106) of an insertion tool (e.g., insertion tool 1105). In some embodiments, the tip 200 may have a tapered shape so as to facilitate insertion of the sleeve and the tip disposed thereon into animal tissue. In some embodiments, the tip 200 may include a generally conical surface 202 having a circular base 212 and a blunt tip 204 at its vertex. In some non-limiting embodiments, the blunt tip 204 may be a rounded tip. In some non-limiting embodiments, the tip 200 may have a shape that is similar to that of a blunted tip 1109 on the distal end of the rod 1104 of the tunneling tool 1103.

In some embodiments, on an end of the tip 200 opposite the rounded tip 204, the tip 200 may be configured to be removably attached to the distal end 1115 of the sleeve 1106. In one embodiment, this may be achieved by a circular skirt, or ring, 208 extending axially, with respect to the longitudinal axis of the conical surface 202, from the base 212 of the conical surface 202. Skirt 208 has a diameter that is smaller than that of the base 212 of the conical surface 202, thereby forming an annular shoulder 210 extending radially with respect to the longitudinal axis of the conical surface 202.

In some alternate embodiments, the tip 200 may be configured so that the distal end 1115 of the sleeve 1106 can be slidably inserted into the end of the tip, for example, by providing an opening at the end of the tip 200 having an inside diameter that is the same as or slightly larger than the outside diameter of the sleeve 1106 at its distal end 1115.

In some embodiments, as shown in FIGS. 7 and 8, a sensor 100 may be inserted into the distal end 1115 of an extended sleeve 1106, and the tip 200 may be placed onto the distal end 1115 of the sleeve 1106 of the insertion tool 1105. In some embodiments, as shown in FIG. 8, which is a transverse cross-section of a tip 200 installed on a sleeve 1106, the axial skirt 208 may be inserted into the sleeve 1106 at the distal end 1115, and the annular shoulder 210 may bear against the distal end 1115 of the sleeve 1106. In some non-limiting embodiments, the outer diameter of the axial skirt 208 may be somewhat smaller than the internal diameter of the sleeve 1106 at its distal end 1115 so that the tip can be inserted into the sleeve 1106 and removably retained there by a friction fit. However, this is not required, and, in some alternative embodiments, the tip 200 may be removably attached to the sleeve 1106 by other means, such as, for example and without limitation, adhering or clipping the tip 200 on the sleeve 1106. In some non-limiting embodiments, the diameter of the base 212 of the conical surface 202 may be substantially the same as the outside diameter of the sleeve 1106 at its distal end 1115, thereby providing a smooth transition between the tip 200 and the sleeve 1106.

In some embodiments, as illustrated in FIG. 8, the sensor 100 may be retained within a compartment at the distal end 1115 of the sleeve 1106. In some non-limiting embodiments, an end 102 of the sensor 100 may project axially beyond the distal end 1115 of the sleeve 1106. Tip 200 may include an internal cavity 206 configured to receive the end portion 102 of the sensor 100, including the portion projecting from the distal end 1115 of the sleeve 1106. In some embodiments, tip 200 may also include an additional hollowed portion 214 extending toward the rounded tip 204.

In one embodiment, the sleeve 1106 may include one or more holes, slots, slits, or other openings to permit the passage of gas, such as EtO gas (the sterilant), and/or a hydrating fluid, such as water, into the sleeve 1106 to contact the sensor 100 disposed therein.

With the tip 200 disposed at the distal end 1115 of the sleeve 1106, the insertion tool can be used as a tunneling tool for opening a subcutaneous pocket in the tissue. When the sleeve 1106 is advanced into the tissue by a specified amount, e.g., as determined by indicator elements, such as bands 1111, 1112, the sleeve 1106 may be retracted, for example, by actuating a slide button (e.g., slide button 1110 (FIG. 3)) coupled to the sleeve 1106, thereby separating the distal end 1115 of the sleeve 1106 from the axial skirt 208 of the tip 200 to thereby release the tip 200 from the sleeve 1106. Further retraction of the sleeve 1106 exposes the sensor 100 and deposits the sensor 100 into the pocket formed by the tip 200 and sleeve 1106. In some embodiments, upon withdrawal of the sleeve 1106 from the pocket, the sensor 100 and the tip 200 may be deposited within the subcutaneous pocket.

In some embodiments, the tip 200 may be bioresorbable and/or biodegradable so that the tip 200 will eventually be dissolved within the tissue. Thus, in some embodiments, the tip may be formed from a material that is biocompatible and produces breakdown products, if any, that are non-toxic. In some embodiments, the tip 200 may be of sufficient strength to withstand the forces applied during insertion and opening of the subcutaneous pocket. In some non-limiting embodiments, the material of which the tip 200 is made may additionally or alternatively be manufactureable, be able to withstand sterilization, and/or have a breakdown half-life of a sufficient duration (e.g., at least 10 minutes), as the sensor may or may not need to be hydrated for at least this length of time prior to insertion.

In some embodiments, the tip 200 may be made of a bioresorbable/biodegradable material, such as a polymer. In some non-limiting embodiments, the polymer may comprise, for example and without limitation, lactic acid, glycolic acid, glucose, polytrimethylene carbonate, collagen, laminin, hydroxyapatite, hyaluronan, and/or amino acids. However, this is not required, and, in some alternative embodiments, the bioresorbable/biodegradable polymer may comprise one or more linear polyesters such as, for example and without limitation, polycaprolactone (PCL), poly(ester-ether)s such as polydioxanone (PDX), poly(amino acid)s such as poly(L-glutamate), poly(L-lysine, poly(L-leucine), poly(anhydride)s such as poly(sebacic acid) and poly(sebacic acid-co-carboxyphenoxypropane) (p(SA-CPP)) ranging all percent SA from 0-100%, including all derivatives, copolymers, block polymers, and blending of these materials. Blending of these polymers will yield various degrees of hardness in the tip 200, different dissolution times, and various and different by-products. As noted above, in some embodiments, the tip 200 may be cored out (hollow), as at internal cavity 214. In some non-limiting embodiments, the tip 200 may be cored out to remove as much excess material as is possible for volume reduction to limit the amount of material that must be dissolved and absorbed and to limit the amount of by-products produced while maintaining strength.

In some embodiments, the tip 200 may be made of a material (e.g., polymer) that constitutes or contains an immune suppressant, such as, for example, poly-salicylate.

In some embodiments, the tip 200 may be made of a material (e.g., polymer) that is combined with a corticosteroid for immune suppression.

In some embodiments, the tip 200 may be made of a material (e.g., polymer) that is combined with angiogenic factors to promote vascular growth and healing.

While the present invention has been described and shown in considerable detail with reference to certain illustrative embodiments, including various combinations and sub-combinations of features, those skilled in the art will readily appreciate other embodiments and variations and modifications thereof as encompassed within the scope of the present invention. Moreover, the descriptions of such embodiments, combinations, and sub-combinations is not intended to convey that the inventions requires features or combinations of features other than those expressly recited in the claims. Accordingly, the present invention is deemed to include all modifications and variations encompassed within the spirit and scope of the following appended claims.

1. An apparatus for implanting an item in animal tissue, said apparatus comprising:
a tubular sleeve comprising a compartment at a distal end of the sleeve, wherein the compartment is configured to receive the item to be implanted such that the item is disposed within the sleeve;
a tapered tip configured to be removably attached to the distal end of said sleeve and to facilitate advancement of an assembly comprising said sleeve and said tip into or through the animal tissue; and
an actuating mechanism coupled to said sleeve and configured to retract said sleeve with respect to the item disposed within the sleeve to thereby remove the item from the sleeve and separate the tip from the distal end of said sleeve.

2. The apparatus of claim 1, wherein the sleeve is made from stainless steel or polytetrafluoroethylene (PTFE).

3. The apparatus of claim 1, wherein said tip has a conical shape.

4. The apparatus of claim 3, wherein said tip has a rounded tip.

5. The apparatus of claim 1, wherein said tip has a ring projecting from an end thereof, and said ring is configured to be inserted into the distal end of said sleeve to retain said tip to said sleeve by a friction fit.

6. The apparatus of claim 1, wherein the tip is configured to be removably attached to the distal end of said sleeve by clipping or adhering the tip to the sleeve.

7. The apparatus of claim 1, wherein the item comprises a sensor configured to detect an analyte or substance of interest in the animal tissue.

8. The apparatus of claim 1, wherein the tip is at least partially hollow.

9. The apparatus of claim 1, wherein the tip is formed from a material that is bioresorbable or biodegradable.

10. The apparatus of claim 1, wherein the tip is made from a polymer comprising one or more materials selected from the group consisting of lactic acid, glycolic acid, glucose, polytrimethylene carbonate, collagen, laminin, hydroxyapatite, hyaluronan, and amino acids.

11. The apparatus of claim 1, wherein the tip is made from a polymer comprising one or more materials selected from the group consisting of polycaprolactone (PCL), poly(ester-ether)s, poly(amino acid)s, and poly(anhydride)s.

12. The apparatus of claim 11, wherein the poly(ester-ether)s comprises polydioxanone (PDX).

13. The apparatus of claim 11, wherein the poly(amino acid)s comprises poly(L-glutamate), poly(L-lysine, or poly(L-leucine).

14. The apparatus of claim 11, wherein the poly(anhydride)s comprises poly(sebacic acid) or poly(sebacic acid-co-carboxyphenoxypropane).

15. The apparatus of claim 9, wherein the material of which the tip is made further comprises an immune suppressant.

16. The apparatus of claim 15, wherein the immune suppressant comprises poly-salicylate or a corticosteroid.

17. The apparatus of claim 9, wherein the material of which the tip is made further comprises angiogenic factors to promote vascular growth and healing.

18. In connection with a device for implanting an item in animal tissue, the device comprising a tubular sleeve and an actuating mechanism, wherein the tubular sleeve comprises a compartment at a distal end of the sleeve, the compartment is configured to receive the item to be implanted such that the item is disposed within the sleeve, and the actuating mechanism is coupled to the sleeve and configured to retract the sleeve with respect to the item disposed within the sleeve to thereby remove the item from the sleeve, a tip comprising:
a tapered end; and
a connecting end opposite the tapered end, wherein the connecting end is configured to enable the tip to be removably attached to the distal end of the sleeve,
wherein the tip is configured such that, when the actuating mechanism retracts the sleeve with respect to the item, the retraction removes the item from the sleeve and also separates the tip from the distal end of the sleeve, and
wherein the tip is formed from a material that is bioresorbable or biodegradable.

19. The tip of claim 18, wherein the connecting end comprises a ring projecting from the end of the tip opposite the tapered end, and said ring is configured to be inserted into the distal end of the sleeve to retain the tip to the sleeve by a friction fit.

20. The tip of claim 18, wherein the tip is configured to be removably attached to the distal end of said sleeve by clipping or adhering the tip to the sleeve.

21. The tip of claim 18, wherein said tip has a conical shape.

22. The tip of claim 21, wherein said tip has a rounded tip.

23. The tip of claim 18, wherein the tip is at least partially hollow.

24. The tip of claim 18, wherein the tip is made from a polymer comprising one or more materials selected from the group consisting of lactic acid, glycolic acid, glucose, polytrimethylene carbonate, collagen, laminin, hydroxyapatite, hyaluronan, and amino acids.

25. The tip of claim 18, wherein the tip is made from a polymer comprising one or more materials selected from the group consisting of polycaprolactone (PCL), poly(ester-ether)s, poly(amino acid)s, and poly(anhydride)s.

26. The tip of claim 25, wherein the poly(ester-ether)s comprises polydioxanone (PDX).

27. The tip of claim 25, wherein the poly(amino acid)s comprises poly(L-glutamate), poly(L-lysine, or poly(L-leucine).

28. The tip of claim 25, wherein the poly(anhydride)s comprises poly(sebacic acid) or poly(sebacic acid-co-carboxyphenoxypropane).

29. The tip of any claim 18, wherein the material of which the tip is made further comprises an immune suppressant.

30. The tip of claim 29, wherein the immune suppressant comprises poly-salicylate or a corticosteroid.

31. The tip of claim 18, wherein the material of which the tip is made further comprises angiogenic factors to promote vascular growth and healing.

32. A method for implanting an item in animal tissue, said method comprising:
placing the item within a tubular sleeve at a distal end of the sleeve;
removably attaching a tapered tip to the distal end of the sleeve;
inserting the tip into an incision formed in the animal tissue;
advancing an assembly comprising the tip and the sleeve with the item disposed therein into the animal tissue, thereby forming a pocket within the animal tissue; and
effecting a retraction of the sleeve with respect to the item within the pocket, thereby removing the item from the sleeve and separating the tip from the distal end of the sleeve and depositing the item and the tip in the pocket within the animal tissue.

33. The method of claim 32, wherein the tapered tip is made of a material that is bioresorbable or biodegradable.
